(19) United States
(12) Patent Application Publication
(10) Pub. No.: US 2009/0012422 A1
(43) Pub. Date: Jan. 8, 2009

(54) Bioptome

(76) Inventor: Eduardo Marban, Beverly Hills, CA (US)

Correspondence Address: BANNER & WITCOFF, LTD., 1100 13th STREET, N.W., SUITE 1200, WASHINGTON, DC, 20005-4051, US

(21) Appl. No.: 11/667,020

(22) PCT Filed: Nov. 8, 2005
(86) PCT No.: PCT/US05/40361

§ 371 Date: Jan. 15, 2008

Related U.S. Application Data
(60) Provisional application No. 60/625,695, filed on Nov. 8, 2004.

(51) Int. Cl. A61B 10/06

(52) U.S. Cl. 600/564

(57) ABSTRACT

A bioptome (1) includes a flexible catheter (2) which can be remotely steered. Two handles (6,7) coupled to a proximal end of the catheter are used to actuate a pair of jaws (4,5) coupled to a distal end of the catheter. A separate control handle (8) includes a rotatable knob (16). Rotation of the knob in one direction causes a distal end of the catheter to bend in a first direction. Rotation of the knob in an opposite direction causes the catheter to bend in a second direction. The control knob may be coupled to the distal catheter end by steering wires (42,43) that move within lumens (44,45) of the catheter in response to knob rotation. The jaws or other operating members) at the distal catheter end may be replaceable (80A, 80B).

### CROSS-REFERENCE TO RELATED APPLICATIONS

This application claims priority to Provisional U.S. Patent Application Ser. No. 60/625,695, titled “Cardiac Stem Cells” and filed Nov. 8, 2004. The contents of said application are incorporated by reference herein in their entirety.

### BACKGROUND

Bioptomes are medical devices which can be used to retrieve tissue samples from internal regions of an animal's body. Known bioptomes consist of a catheter having a pair of jaws on a distal end. The jaws typically have sharpened edges for cutting into tissue, and an interior cavity for retaining a cut piece of tissue when the jaws are closed. A pair of handles at the proximal end of the catheter is used to open and close the jaws. In use, the distal end of the catheter is typically inserted into a blood vessel of a human patient (or of an animal) from which a tissue sample is desired. The jaws are then pushed to the body region from which a tissue sample is needed. Using the handles at the proximal end of the catheter, the jaws are manipulated and a piece of tissue removed. The catheter is then pulled from the body and the tissue sample retrieved from the jaws.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In at least some embodiments, a bioptome includes a flexible catheter which can be remotely steered. Two handles coupled to a proximal end of the catheter are used to actuate a pair of jaws coupled to a distal end of the catheter. A separate control handle includes a rotatable knob. Rotation of the knob in one direction causes a distal end of the catheter to bend in a first direction. Rotation of the knob in an opposite direction causes the catheter to bend in a second direction. In some embodiments, the control knob is coupled to the distal catheter end by steering wires. The steering wires move within lumens of the catheter in response to knob rotation, and effect bending by pulling upon the distal end in a particular direction. In yet other embodiments, the jaws or other operating member(s) at the distal catheter end are replaceable.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 shows a bioptome according to at least some embodiments.

FIG. 2 shows opening of the jaws of the bioptome of FIG. 1.

FIGS. 3 and 4 show movement of a distal catheter portion of the bioptome of FIG. 1 in response to movement of a control knob.

FIG. 5 is an enlarged view of the portion of the bioptome indicated in FIG. 1, and shows additional details of bioptome jaws according to at least some embodiments.

FIG. 6 is another view, from the location indicated in FIG. 5, of a portion of the bioptome of FIG. 1.

FIG. 7 is a cross-sectional view, from the location indicated in FIG. 6, of a portion of the bioptome of FIG. 1.

FIG. 8 is an enlarged view, from the location indicated in FIG. 1, of the control handle of the bioptome of FIG. 1.

FIG. 9 is a cross-sectional view, from the location indicated in FIG. 8, of the control handle of FIG. 8.

FIG. 10 is an enlarged view of a portion of the bioptome of FIG. 1, and shows opening of the jaws.

FIGS. 11 and 12 are enlarged views of a portion of the bioptome of FIG. 1, and show bending of the bioptome catheter in response to remote manipulation of a control knob.

FIG. 13 is an enlarged view of a distal catheter end and of a replacement jaw unit according to at least some embodiments.

FIG. 14 is a cross-sectional view of a catheter for a bioptome according to at least one alternate embodiment.

### DETAILED DESCRIPTION

Known bioptomes have several disadvantages. For example, existing bioptomes are relatively stiff. Typically, the catheters of such bioptomes have a limited flexibility that is similar to the flexibility of a wire coat hanger. This limited flexibility can sometimes prevent the bioptome catheter from reaching certain regions inside a body. For example, certain regions may only be reachable through blood vessels and/or other passages that contains turns bending at relatively large angles. A conventional bioptome catheter unable to bend at those angles cannot reach the desired regions without injuring the patient.

Apart from their relative stiffness, the non-steerable nature of existing bioptomes limits the regions from which tissue samples can be retrieved. Specifically, a physician using an existing bioptome cannot change the shape of the distal catheter end once it is inside the patient's body. Instead, the catheter end tends to follow along the shape of the internal body cavity in which it is located. If a physician could curve the catheter distal end using a remote control located outside of a patient's body, the physician could more accurately guide the catheter to a desired location through various blood vessel branches, etc. A remotely guidable catheter could also permit a physician to bend the distal end to reach a tissue location within a particular body cavity without having to push the jaws along the contour of that body cavity.

Because of recent developments, there is now a motivation to obtain tissue samples from regions not generally accessible with existing bioptomes. As described in Provisional U.S. Patent Application Ser. No. 60/625,695, titled “Cardiac Stem Cells” and filed Nov. 8, 2004, cardiac stem cells harvested from a patient with a cardiac bioptome can be cultured and then therapeutically reintroduced into the patient's heart. Because of their stiffness and lack of steerability, cardiac bioptomes are generally limited to obtaining cells from a few specific regions of a heart (typically, the right ventricular septum). However, it is desirable to harvest cardiac stem cells from additional areas such as (but not limited to) the crista terminalis, the left and right atrial appendages, and the atrioventricular groove. A guidable and more flexible bioptome would permit retrieval of tissue from such regions.

Another disadvantage of known bioptomes pertains to the sharpness of the jaws. Existing bioptomes are designed for reuse in multiple patients, and thus require frequent sterilization (e.g., autoclaving). As a result of repeated sterilization and reuse, the cutting edges of the jaws tend to become dull. Dull jaws tend to pull on the interior surface of the heart when obtaining a tissue sample, potentially causing injury to the harvested tissue or to the patient. At least some of the regions from which tissue samples might be desired for cardiac stem cell harvesting are thinner than the areas biopsied with conventional bioptomes. Use of dull jaws in such a region could pose a higher risk of serious injury to the patient.

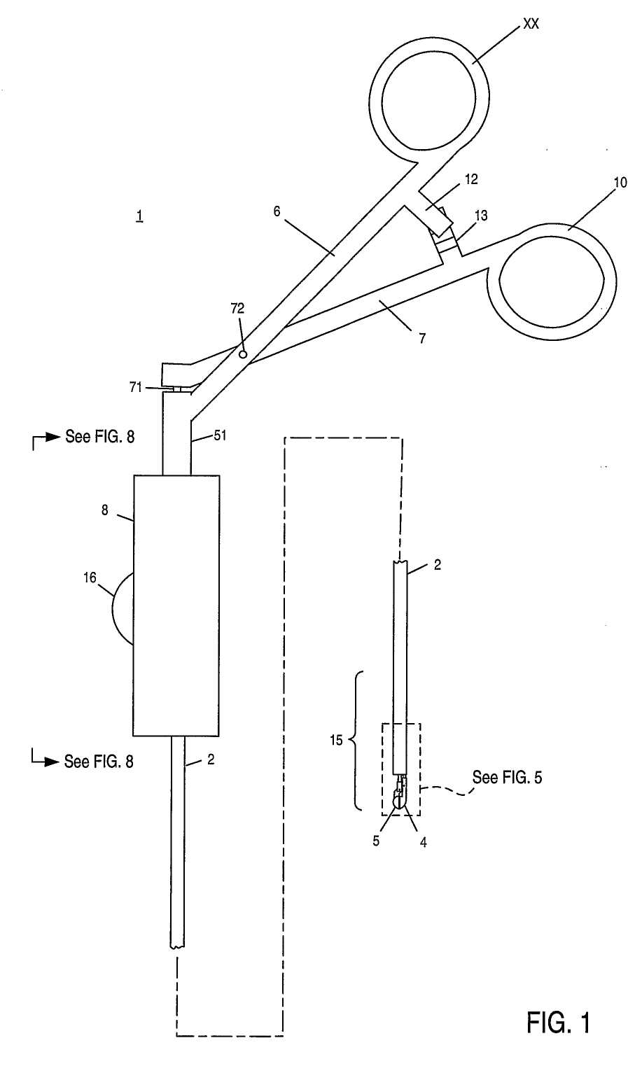

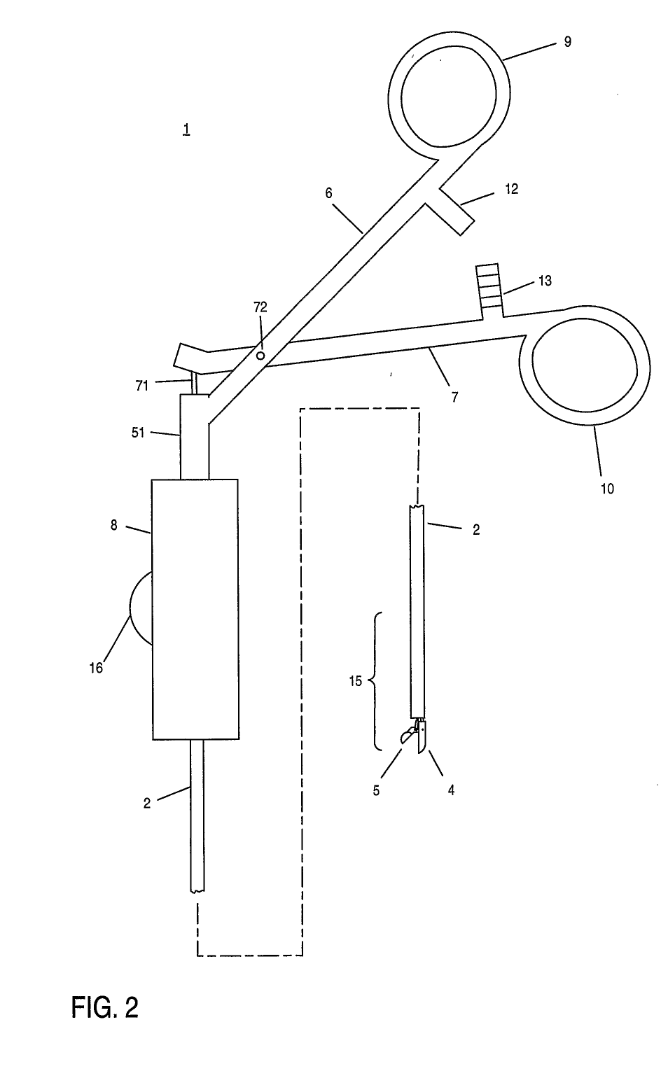

In at least some embodiments, a steerable and more flexible bioptome having replaceable (and/or disposable) jaws avoids many of the disadvantages of existing devices. FIG. 1 shows a bioptome 1 according to at least some embodiments. Similar to existing devices, bioptome 1 includes a catheter 2. Disposable jaws 4 and 5 are attached to a distal end of catheter 2. Additional details of jaws 4 and 5 and of their attachment to catheter 2 are provided below. Attached to a proximal end of catheter 2 are jaw actuation handles 6 and 7 and a control handle 8. Handles 6 and 7 include finger loops 9 and 10 and ratchet tabs 12 and 13. As with conventional bioptomes, and as seen in FIG. 2, separation of finger loops 9 and 10 causes jaws 4 and 5 to open. When finger loops 9 and 10 are pulled together, jaws 4 and 5 are closed. Tabs 12 and 13 engage and hold handles 6 and 7 in the closed configuration.

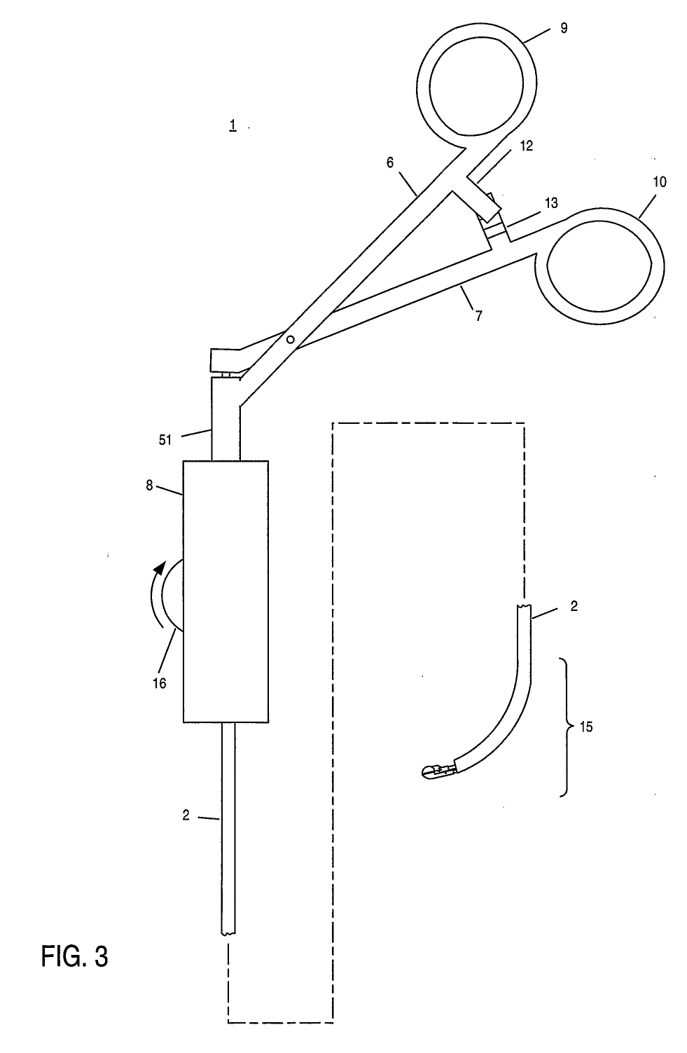

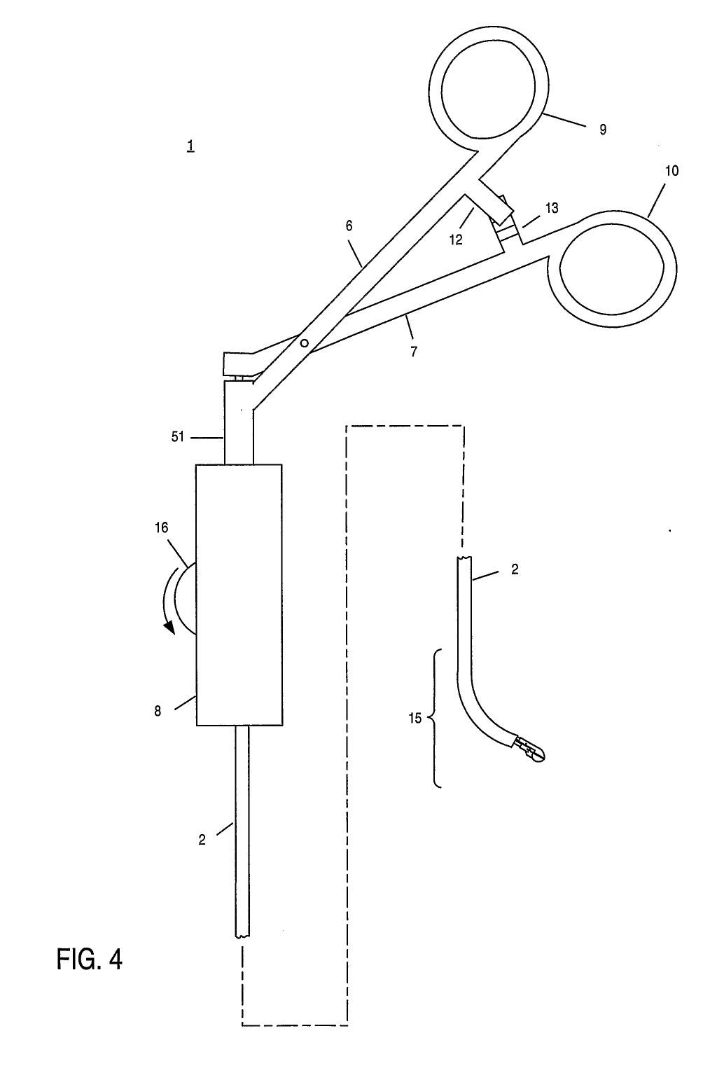

Unlike existing bioptomes, the distal end of bioptome 1 is more flexible. Moreover, a distal portion 15 of catheter 2 can be remotely bent by a physician using control handle 8. In particular, and as seen in FIG. 3, rotating knob 16 of control handle 8 in one direction causes distal portion 15 to bend in one direction. Conversely, moving knob 16 in the opposite direction causes distal portion 15 to bend in another direction (FIG. 4).

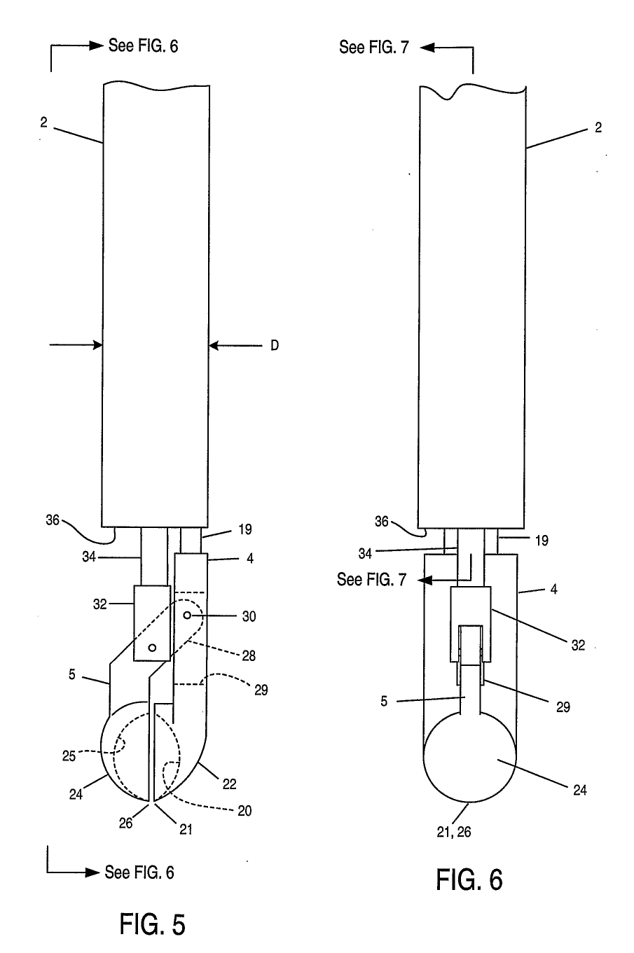

FIG. 5 is an enlarged view of the portion of bioptome 1 indicated in FIG. 1, and shows additional details of jaws 4 and 5. FIG. 6 is a view of jaws 4 and 5 and of catheter 2 from the position indicated in FIG. 5. In at least some embodiments, bioptome 1 is sized for cardiac applications, and the diameter (D) of catheter 2 is between approximately two to five millimeters. In some embodiments, diameter D is between four and five millimeters. One end of fixed jaw 4 is coupled to an attachment post 19 on the distal end of catheter 2. As used herein, “coupled” includes two components that are attached (movably or fixedly) directly or by one or more intermediate components. The other end of fixed jaw 4 has a round end 22 with an internal hemispherical cup 20 formed therein. Cup 20 is shown in broken lines in FIG. 5; for simplicity, broken lines for cup 20 are omitted from subsequent drawing figures. A leading edge 21 of cup 20 is sharpened so as to permit cutting and removal of tissue.

Hinged jaw 5 is attached to fixed jaw 4, and includes a rounded end 24 with an internal hemispherical cup 25, as well as a sharpened leading edge 26. As with cup 20 on fixed jaw 4, cup 25 is shown in broken lines in FIG. 5, with those broken lines similarly omitted from other drawing figures for simplicity. The other end 28 of hinged jaw 5 rests within a slot 29 in fixed jaw 4. Hinged jaw 5 pivots about a pin 30 securing end 28 within slot 29. A clevis 32 is pivotally attached to hinged jaw 5 and to jaw actuation cable 34. When cable 34 is pulled (by spreading of finger loops 9 and 10, as shown in FIG. 2), cable 34 pulls clevis 32 toward end cap 36 of catheter 2. Hinged jaw 5 thereby pivots about pin 30 and opens jaws 4 and 5. Similar to existing bioptomes, fixed jaw 4 and hinged jaw 5 can then be brought together (by squeezing together finger loops 9 and 10) to pinch and cut a piece of tissue. The cut tissue is retained in a cavity formed by cups 20 and 25. As with cups 20 and 25, slot 29 and portions of hinged jaw 5 are shown in broken lines in FIG. 5. For simplicity, these broken lines are similarly omitted in subsequent drawing figures.

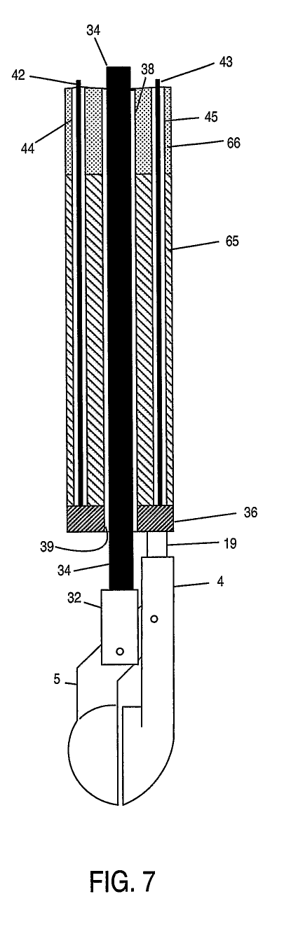

FIG. 7 is a cross-sectional view of the portion of catheter 2 indicated in FIG. 6. The distal end of catheter 2 includes an end cap 36. Jaw actuation cable 34 moves within a central lumen 38 and extends through an orifice 39 in end cap 36. A seal (not shown) surrounds cable 34 at orifice 39. The seal allows cable 34 to slide in and out of orifice 39, but prevents blood and other fluid from entering lumen 38 from the patient, and also prevents contaminates from entering the patient via lumen 38. As also shown in FIG. 7, steering wires 42 and 43 rest within lumens 44 and 45 and are attached to end cap 36. As explained in more detail below, wires 42 and 43 are pulled to effect bending of distal portion 15 as shown in FIGS. 3 and 4.

In some embodiments, and as shown in FIG. 7, distal portion 15 may include materials having different degrees of stiffness. A first region 65 is more distally located along the longitudinal axis of catheter 2 than a second region 66. Region 65 is formed (at least in part) from a material that is substantially more flexible than a material from which region 66 is formed (in whole or in part). In this manner, and as described in more detail below, pulling of wire 42 or wire 43 causes region 65 to bend more than region 66. This permits design of catheter 2 so as to control where bending will occur along the catheter length in response movement of control knob 16.

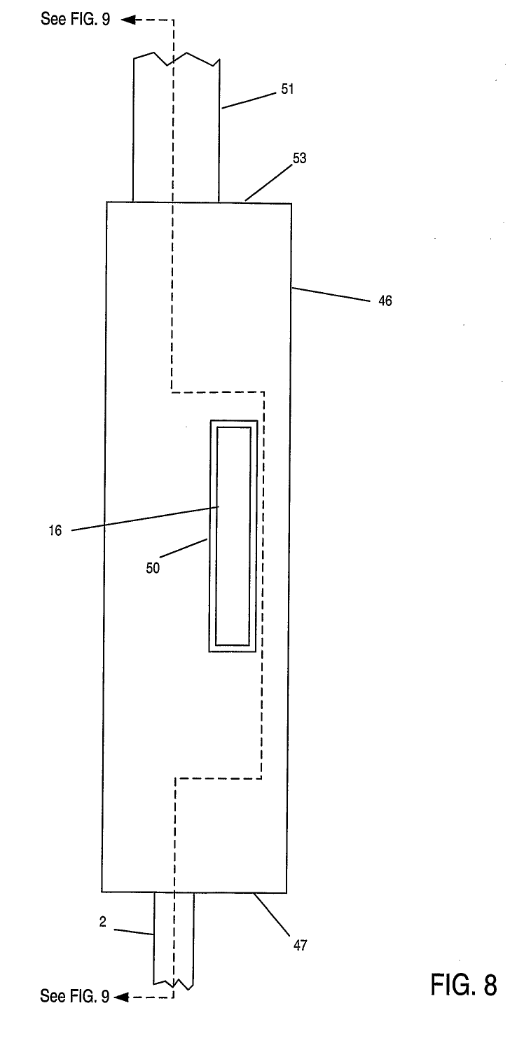

FIG. 8 is an enlarged view of control handle 8 from the position indicated in FIG. 1. As seen in FIG. 8, knob 16 extends through an opening 50 in a housing 46 of control handle 8. Catheter 2 enters housing 46 at a lower end 47. Tube 51 (attached to handle 6) is attached to housing 46 at an upper end 53.

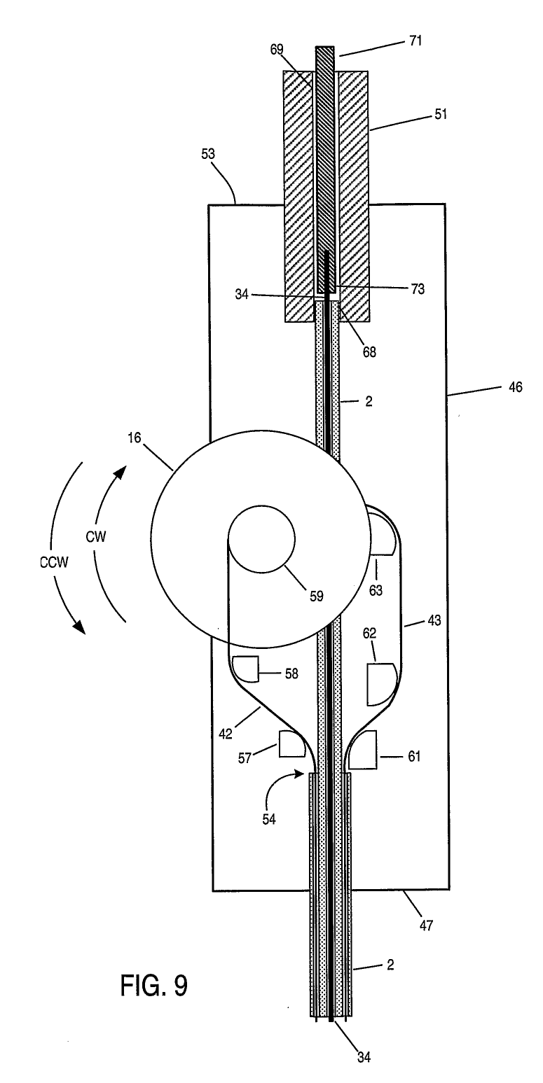

FIG. 9 is a partially schematic cross-sectional view of control handle 8 taken from the position shown in FIG. 8. A portion of the outer surface of catheter 2 is attached to housing 46 near lower end 47 so as to be immovable relative to housing 46. However, steering wires 42 and 43 remain movable. In particular, and as seen in FIG. 9, wires 42 and 43 exit catheter 2 at region 54 in which the outer portions of catheter 2 have been removed. Wire 42, after routing through guides 57 and 58, wraps around a pulley 59 that is concentric with (but smaller in diameter than) control knob 16. Wire 43, after routing through guides 61, 62 and 63, wraps around a similar pulley (not seen in FIG. 9) on an opposite side of knob 16. Wire 42 is wrapped around a pulley 59 such that rotation of knob 16 in the “CW” direction causes wire 42 to be pulled within lumen 44 in the proximate direction. Wire 43 is wrapped in an opposite direction around the pulley on the other face of knob 16. Accordingly, rotation of knob 16 in the “CCW” direction causes wire 43 to be pulled within lumen 45 in the proximate direction.

As also seen in FIG. 9, a terminal portion 68 of catheter 2 that remains after separation of wires 42 and 43 is attached to a lower end of tube 51 inside of a bore 69. So as to avoid interference with the operation of knob 16, knob 16 is offset outward from catheter 2 in the plane FIG. 9. This offset is generally seen in FIG. 8. Wires 42 and 43 thus extend slightly outward from the plane of FIG. 9 as they go from region 54 to pulleys on knob 16. The outer surface of the terminal end 68 is immovably fixed to tube 51. However, rod 71 moves within bore 69 in response to movement of handle 7 (see FIG. 2) relative to handle 6 about pivot 72. Bottom end 73 of rod 71 is attached to an exposed end of jaw actuation cable 34 that emerges from central lumen 38. As rod 71 moves upward, jaw actuation cable 34 is pulled from lumen 38. As rod 71 moves downward, cable 34 is pushed in the opposite direction.

Although a knob is shown as the control member in control handle 8, other types of control members could be used. Examples include a slide, a lever, and a control wheel. Coupling of these and other alternative control members to steering wires is within the routine ability of a person of ordinary skill in the art once such a person is supplied with the information provided herein. Although the description herein refers to “cables” and “wires,” other types of tensile-force-transferring flexible members could be used.

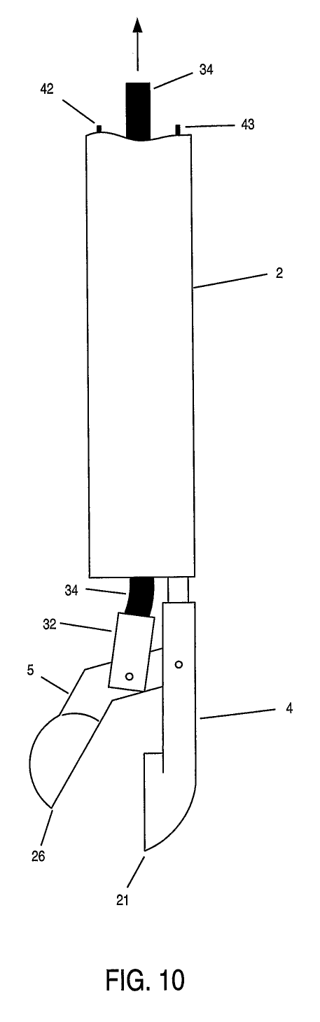

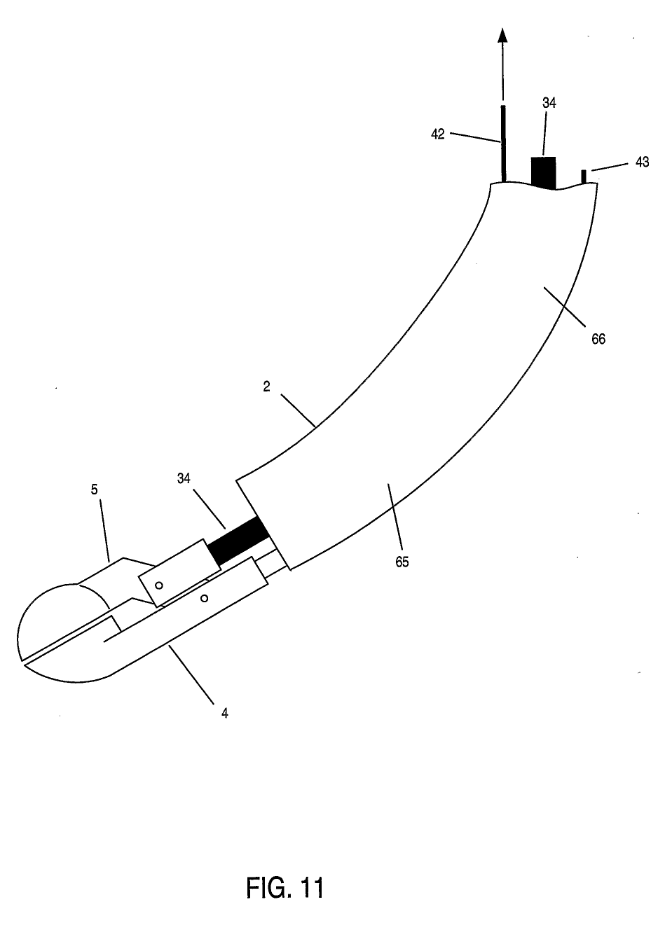

Operation of bioptome 1 is further illustrated in FIGS. 10-12. FIG. 10 shows pulling of jaw actuation cable 34 in response to movement of handle 7 relative to handle 6. Cable 34 pulls on clevis 32, which in turn pulls upon hinged jaw 5. In response to this pull, jaw 5 pivots about pin 30 into an open configuration. When handles 6 and 7 are pulled together, cable 34 moves in the opposite direction and pushes hinged jaw 5 into a closed configuration.

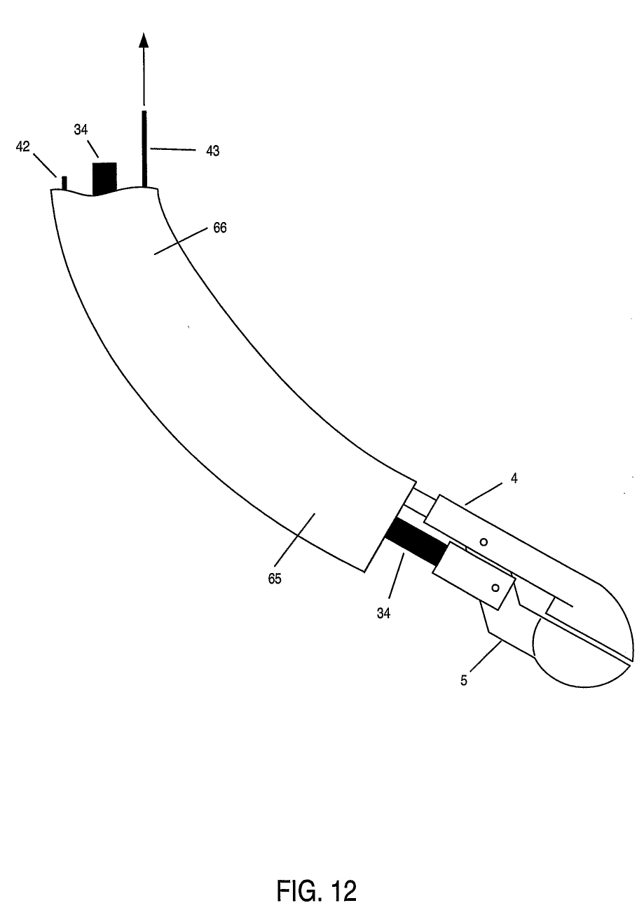

FIG. 11 illustrates bending of distal portion 15 of catheter 2 in response to rotation of knob 16 clockwise in FIG. 9. As knob 16 rotates clockwise, wire 42 is pulled in the direction indicated in FIG. 11. In response to the shortening of cable 42 on one side of catheter 2, distal end cap 36 is pulled toward that side. Because the material in region 65 is more flexible than the material in region 66, the distal end of catheter 2 bends in the manner shown. Conversely, rotating knob 16 counterclockwise (FIG. 9) pulls wire 43 in the direction indicated in FIG. 12. In response to the shortening of wire 43 on that side of catheter 2, end cap 36 is pulled to thereby cause bending as shown in FIG. 12.

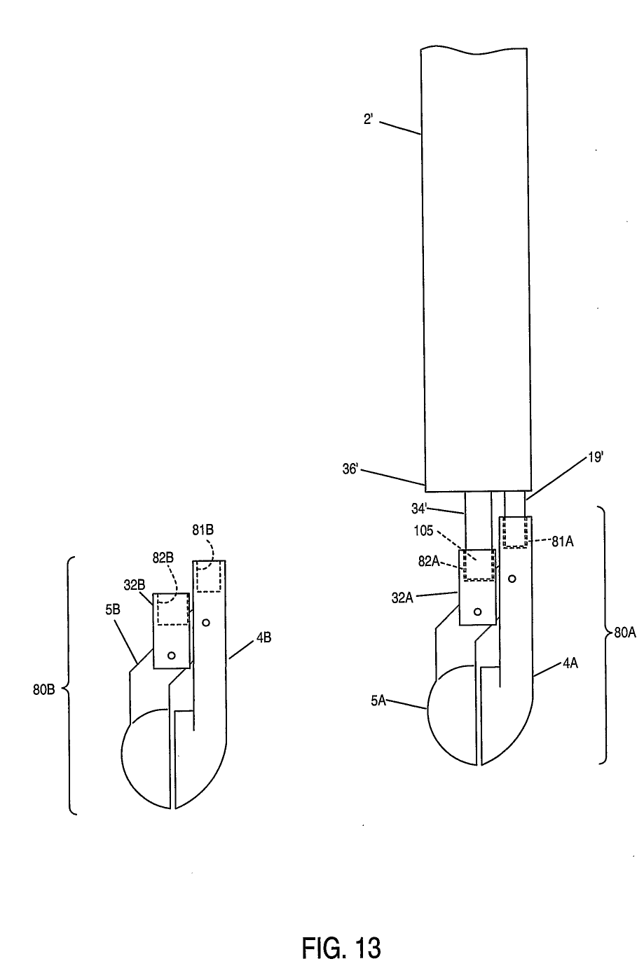

FIG. 13 illustrates another aspect of at least some embodiments of the invention. In the embodiment of FIG. 13, disposable jaw unit 80A (which includes fixed jaw 4A and hinged jaw 5A) is removable from catheter 2′ (without damage to catheter 2′) and replaceable with disposable jaw unit 80B. An extension 19′ from end cap 36′ fits into a receiving recess 81A in fixed jaw 4A. Similarly, the distal terminal end 105 of cable 34′ fits within a receiving recess 82A in clevis 32A. Upon removal of jaw unit 80A from catheter 2′, extension 19′ and cable end 105 may be respectively placed into recesses 81B and 82B of jaw unit 80B. For simplicity, recesses 81A, 81B, 82A and 82B, a portion of extension 19′ and cable end 105 are shown as simple rectangular shapes. In practice, however, jaw units 80A and 80B would be securely attached to catheter 2′ so as to prevent unwanted detachment while the bioptome is use. Numerous known mechanisms for securely attaching components can be employed. The selection and application of such mechanisms is within the routine abilities of a person of ordinary skill in the art (once such a person is provided with the information provided herein). In some embodiments, for example, extension 19′ is threaded. Extension 19′ is screwed into mating threads in recess 81A (or 81B), and cable end 105 (which is fitted with a ball or other similar fixture) is snap-fit into recess 82A (or 82B).

In at least some embodiments, a jaw assembly is replaceable with a jaw assembly that is not identical to the assembly being replaced. For example, a second type of jaw assembly may be modified to obtain samples from tissue that is more delicate than a type of tissue from which a first type of jaw assembly is designed to obtain samples. The second jaw assembly might be smaller and/or have sharpened edges that are of a different shape (e.g., more blunt or more pointed). Moreover, a jaw assembly might be replaceable with a device other than a jaw assembly. Except as specifically recited in a particular claim, however, the invention is not limited by the specific type of jaw assembly attached (or attachable) to a bioptome catheter.

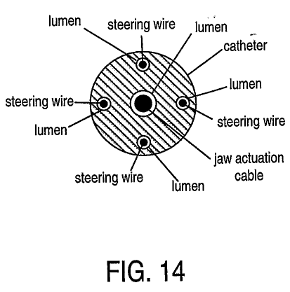

In yet other embodiments, a bioptome is steerable in more than two directions. In at least some such embodiments, the catheter includes an additional set of lumens similar to lumens 44 and 45 shown in FIG. 7, but positioned as shown in FIG. 14. FIG. 14 is a cross-sectional view of a catheter according to an alternate embodiment in which the distal catheter end can be remotely controlled to bend in four directions. As seen in FIG. 14, a central lumen houses a jaw actuation cable. Four lumens near the periphery of the catheter house steering wires. Similar to the embodiment of FIG. 7, those steering wires are attached to an end cap (or other structure) at or near the distal catheter end. Two of the steering wires are attached to a first control knob (or other control member) in a control handle at or near a proximal end of the bioptome catheter, and the other two steering wires are attached to a second knob (or other type of control mechanism) in the control handle. The physician can remotely bend the distal catheter end in any of four directions by manipulating the control knobs at the proximal end.

In some embodiments, a bioptome such as is described above is used to obtain samples of tissue from within a patient's heart. Under local anesthesia, an intravenous line (e.g., a sheath or cannula) is placed into the jugular vein in the patient's neck. The bioptome is introduced directly into the heart through the intravenous line, and is guided to the desired location using an X-ray device, ultrasound, magnetic resonance imaging, or other type of tracking process. By bending the distal catheter end from the proximally-located control(s), the catheter is steered to the desired location. Tissue samples are taken with the jaws, and the catheter is then withdrawn. This procedure may then be repeated multiple times to obtain additional samples. The invention is not limited to use in the manner described above, however. Moreover, the invention is not limited to use in cardiac regions or in conjunction with cardiac procedures. For example, a bioptome can be used to access a tissue sample from other sources, including but not limited to the kidneys, liver, spleen and pancreas. Indeed, a bioptome according to various embodiments is not limited to use in a human. In some embodiments, a bioptome is used to obtain tissue samples from a non-human animal.

Although examples of carrying out the invention have been described, those skilled in the art will appreciate that there are numerous variations and permutations of the above described devices that fall within the spirit and scope of the invention as set forth in the appended claims. It is to be understood that the subject matter defined in the claims is not necessarily limited to the specific features or acts described above. Rather, the specific features and acts described above are disclosed as example forms of implementing the claims.

1. A bioptome, comprising:
a flexible catheter having first and second lumens formed therein, a proximal end and a distal end;
a first tensile member extending through the first lumen from the proximal end to the distal end;
first and second jaws located at the catheter distal end, at least one of the first and second jaws coupled to the first tensile member and movable in response to movement of the first tensile member within the first lumen; and
a second tensile member movable within the second lumen, wherein a pulling force applied to the second tensile member from the catheter proximate end causes bending in a first direction of a distally-located portion of the catheter.

2. The bioptome of claim 1, wherein a substantial portion of the catheter has an outside diameter of between 2 and 5 millimeters.

3. The bioptome of claim 1, wherein a substantial portion of the catheter has an outside diameter of between 4 and 5 millimeters.

4. The bioptome of claim 1, wherein the catheter has a third lumen formed therein, and further comprising:
a third tensile member movable within the third lumen, wherein a pulling force applied to the third tensile member from the catheter proximate end causes bending of the distally-located portion of the catheter in a second direction.

5. The bioptome of claim 4, further comprising a control handle having a control knob rotatably mounted therein, and wherein:
the second tensile member emerges from the second lumen and is coupled to the control knob,
the third tensile member emerges from the third lumen and is coupled to the control knob,
rotation of the control knob in a first rotary direction pulls the second tensile member within the second lumen, and
rotation of the control knob in a second rotary direction pulls the third tensile member within the third lumen.

6. The bioptome of claim 1, wherein a first longitudinal region of the catheter is stiffer than a second longitudinal region of the catheter.

7. The bioptome of claim 6, wherein the distally-located portion includes the second longitudinal region, and wherein the first longitudinal region is adjacent to the second longitudinal region on a proximal side of the catheter.

8. The bioptome of claim 1, wherein:
the catheter distal end is configured for removal of the first and second jaws without damage to the catheter distal end, and
the catheter distal end is further configured for attachment, after removal of the first and second jaws, of replacement first and second jaws.

9. A bioptome, comprising:
a flexible catheter having a lumen formed therein, a proximal end and a distal end;
a tensile member extending through the lumen from the proximal end to the distal end; and
first and second jaws located at the catheter distal end, at least one of the first and second jaws coupled to the tensile member and movable in response to movement of the tensile member within the lumen, and wherein
the catheter distal end is configured for removal of the first and second jaws without damage to the catheter distal end, and
the catheter distal end is further configured for attachment, after removal of the first and second jaws, of replacement first and second jaws.

10. The bioptome of claim 9, wherein a substantial portion of the catheter has an outside diameter of between 2 and 5 millimeters.

11. A bioptome, comprising:
a flexible catheter having a lumen formed therein, a proximal end and a distal end;
a tensile member extending through the lumen from the proximal end to the distal end;
first and second jaws located at the catheter distal end, at least one of the first and second jaws coupled to the tensile member and movable in response to movement of the tensile member within the lumen; and
a control member coupled to the catheter at a proximal end thereof, wherein movement of the control member causes bending of a distally-located portion of the catheter.

12. The bioptome of claim 11, wherein the control member is a rotating knob.
